(19) United States
(12) Patent Application Publication
(10) Pub. No.: US 2023/0398522 A1
(43) Pub. Date: Dec. 14, 2023

(54) Process And Catalyst For The Catalytic Hydrogenation Of Organic Carbonyl Compounds

(71) Applicant: TOPSOE A/S, Kgs. Lyngby (DK)

(72) Inventors: Poul Erik HØJLUND NIELSEN, Fredensborg (DK); Niels Christian SCHJØDT, Hvalsø (DK); Susanne Lægsgaard JØRGENSEN, Holte (DK); Uffe Vie MENTZEL, Vanløse (DK); Matthias Josef BEIER, Birkerød (DK); Henrik Junge MORTENSEN, Taastrup (DK)

(73) Assignee: TOPSOE A/S, Kgs. Lyngby (DK)

(21) Appl. No.: 18/250,277

(22) PCT Filed: Nov. 24, 2021
(86) PCT No.: PCT/EP2021/082821

§ 371 Date: Apr. 24, 2023

(30) Foreign Application Priority Data
Nov. 24, 2020 (EP) 20209523.8

(51) Int. Cl. B01J 23/80; B01J 21/02; B01J 37/08; B01J 35/10; B01J 35/00; B01J 6/00; B01J 37/03; B01J 21/00; C07C 29/145

(52) U.S. Cl. CPC B01J 23/80; B01J 21/02; B01J 37/08; B01J 35/1033; B01J 35/1004; B01J 35/006; B01J 6/001; B01J 37/03; B01J 35/0026; B01J 21/005; C07C 29/145

(57) ABSTRACT

Process for the catalytic hydrogenation of organic carbonyl compounds containing at least one functional group belonging to the group of aldehydes, ketones, esters and carboxylic acids, whereby said at least one functional group is converted to an alcohol by contacting said carbonyl compound with hydrogen and a hydrogenation catalyst at elevated temperature and pressure as well as a catalyst therefore and a process for producing said catalyst.

### FIELD

The present invention pertains to catalytic hydrogenation in the gas phase or liquid phase of organic carbonyl compounds in the presence of a catalyst comprising Cu, Zn and Al. It also pertains to a method of preparing such a catalyst and to the catalyst obtainable by the method.

### BACKGROUND

Organic carbonyl compounds are those organic compounds which contain at least one C═O group such as aldehydes, ketones, esters and carboxylic acids.

Catalytic hydrogenation of organic carbonyl compounds to their corresponding alcohols is an important reaction in the chemical industry. Aldehydes, ketones, esters and carboxylic acids can be hydrogenated to alcohols. The process is employed for the manufacture of important alcohols such as 1-propanol and 2-propanol, n-butanol and iso-butanol, 2-ethylhexanol, fatty alcohols, various glycols and diols and many more. For many years, it has been common practice in the chemical industry to use catalysts containing environmentally problematic compounds such as chromium and nickel. Although the more benign Cu/Zn/Al catalysts have catalytic activity for these reactions, it has hitherto not been possible to make Cu/Zn/Al catalyst formulations with sufficient mechanical strength, chemical inertness and catalytic activity and selectivity to replace Cr- or Ni-containing catalysts in industrial applications.

A frequently used Cu-based catalyst for hydrogenation of organic carbonyl compounds is Adkins catalyst, often called copper chromite in the industry. The chromium is beneficial for the mechanical strength of the catalyst, but it is an environmental and health concern. Ni-catalysts are also employed in the catalytic hydrogenation of carbonyl compounds to alcohols. Hydrogenation catalysts based on Ni are inherently more active than the Cu-based catalysts but are typically less selective.

Furthermore, nickel compounds may cause allergy and are classified as human carcinogens. In some hydrogenation processes, Cu-catalysts can replace Ni-catalysts provided that the former has sufficient activity, selectivity, mechanical stability and chemical inertness.

U.S. Pat. No. 10,226,760 regards a method for producing a shaped Cu—Zn catalyst for hydrogenating organic compounds containing a carbonyl function. The shaped catalyst is suitable for hydrogenating aldehydes, ketones and also carboxylic acids and/or their esters. It also regards Cu—Zn catalysts obtainable by the production process.

In U.S. Pat. Nos. 5,142,067 and 5,008,235 a process and catalysts are disclosed for hydrogenating organic feeds containing bound oxygen into their corresponding alcohols.

U.S. Pat. No. 6,455,464 discloses a non-chrome, copper-containing catalyst and a method of preparing the same.

Commercially available catalysts of the Cu/Zn/Al kind usually have a high Cu-content and contain significant amounts of free ZnO. These catalysts have low mechanical strength, which precludes their use in hydrogenation reactions. Furthermore, these known catalysts are sensitive to carboxylic acids, since carboxylic acids tend to react with zinc oxide under the reaction conditions, thus deteriorating the catalyst. Furthermore, state of the art Cu/Zn/Al catalysts do not have sufficiently stable activity, resulting in a relatively short catalyst lifetime.

In U.S. Pat. No. 5,142,067 Cu—Al—X catalysts for hydrogenation are disclosed having a high copper content. The third metal may be zinc.

In Shi Zhangping et al., “Effects of the preparation method on the performance of the Cu/ZnO/Al₂O₃ catalyst for the manufacture of L-phenylalaninol with high ee selectivity from L-phenylalanine methyl ester”, Catal. Sci. Technol., vol. 4, 1 Jan. 2014. In Shi et al., Cu/Zn/Al catalyst compositions are produced by fractional coprecipitation which in its oxidized form has no or very little spinel phase. Shi et al. conclude that lower calcination temperatures provide higher copper surface areas which provide higher activities and/or selectivities. A calcination temperature of 450° C. is exemplified. It is evident from their FIG. 2 and from page 1136, column 1 that no spinel phase is present: “The results show that no diffraction peaks of Al₂O₃ and ZnO can be detected, suggesting that Al₂O₃ and ZnO phases are amorphous or highly dispersed.” Accordingly, in the oxidized form of the Shi catalyst, all the Cu is present as CuO. In Shi et al. the coprecipitation methods a) to d) involve the use of aluminum nitrate as the source of aluminum.

EP 0 011 150 discloses a Cu/Zn/Al catalyst for synthesis of methanol.

However, there is still a need for industrially applicable catalyst compositions useful in hydrogenation of biobased feeds and in particular of organic carbonyl compounds present in biobased feeds. The invention also regards the use of potassium or sodium aluminates for preparing catalyst compositions useful in industrial hydrogenation of biobased feeds.

### SUMMARY OF THE INVENTION

The present inventors have developed a novel and improved catalyst composition for catalytic hydrogenation of organic carbonyl compounds. They developed an improved process for producing the catalyst composition which provided an improved internal structure to improve activity, selectivity, stability and mechanical strength without using harmful elements such as nickel or chromium.

According to an aspect of the present invention a catalyst composition is provided for catalytic hydrogenation of an organic carbonyl compound, the composition comprising in its oxidized form 12-38% by weight of Cu, 13-35% by weight of Zn, and 12-30% by weight of Al; and the composition having a molar ratio of Zn:Al in the range 0.24-0.60; and the composition comprising in its oxidized form at least 50% by weight of a spinel structure as determined by X-ray diffraction (XRD).

The catalysts of the present invention are particularly appropriate for hydrogenation of organic carbonyl compounds to their corresponding alcohols. The catalysts obtained according to the present invention comprise, in their active form, metallic Cu and ZnAl₂O₄ as the main components as observed by XRD. An important advantageous feature of the invention is that the catalyst contains, in its active (reduced) form, a limited amount of free zinc oxide. It is characteristic for the catalyst of the present invention that on calcination of the catalyst precursor, a mixed Cu/Zn spinel is formed, which transforms gradually at increasing temperatures in an O₂-containing atmosphere to CuO and ZnAl₂O₄.

The catalysts are additionally characterized by their high activity, selectivity and high mechanical strength and by being free of elements such as chromium and nickel, which are hazardous to human health and the environment. In addition, the catalyst composition according to the invention has an improved catalytic stability in the sense that it retains its hydrogenation activity for a prolonged period of time. All these advantages make the catalyst compositions according to the present invention highly suitable for industrial applications.

The inventors found an improved process for preparing the catalyst according to the invention. They found that combining the features of a new aluminium source with selecting certain relative ranges of copper, zinc and aluminium—upon calcination—resulted in surprisingly good catalysts for use in industrial hydrogenation processes. They also surprisingly found that for the disclosed compositions there is an optimal calcination range—which is higher than expected. The improved characteristics are disclosed throughout the document.

According to another aspect of the present invention a method is provided for preparing an oxidized form of a catalyst composition for catalytic hydrogenation of an organic carbonyl compound comprising the steps of:
a. Coprecipitating:
   I. an acidic solution of salts of Cu and Zn having a Cu:Zn weight ratio in the range of from 0.3 to 2.5; and
   II. a basic solution of an aluminate salt further containing one or more soluble hydroxide salts and one or more soluble carbonate salts;
to obtain a catalyst precursor composition having a molar ratio of Zn:Al in the range of from 0.24 to 0.60;
b. Calcining the catalyst precursor composition at a temperature Tcalc in the range of from 250 to 900° C. to obtain an oxidized form of a catalyst composition for catalytic hydrogenation of an organic carbonyl compound, the catalyst composition comprising in its oxidized form 12-38% by weight of Cu, 13-35% by weight of Zn, and 12-30% by weight of Al, the remainder being mainly oxygen; and the catalyst composition having a molar ratio of Zn:Al in the range of from 0.24 to 0.60; and the catalyst composition comprising in its oxidized form at least 50% by weight of a spinel structure as determined by X-ray diffraction (XRD).

The inventors found that following this method resulted in the improved catalytic compositions described herein. In particular, they found that using alkali aluminate as aluminum source, dissolving it in a basic solution and coprecipitating it with an acidic solution comprising copper and zinc ions, provided an improved precursor which upon calcination at 250-900° C. provided catalyst compositions having much higher amounts of spinel phase than prior art Cu/Zn/Al catalysts. In particular, the inventors found that at lower calcination temperatures in the range of from 250 to 550° C. most copper and zinc would be bound as a mixed spinel of the type CuₓZn₁₋ₓAl₂O₄. The spinel phase may make up as much as above 90% by weight of the catalyst composition as determined by XRD at the lower calcination temperatures in the range of 250-550° C. Without being bound by theory, the inventors hypothesize that an advantage of this is that upon reduction (activation) of the catalyst, the metallic Cu particles forming the active phase in the catalyst are born from copper ions in the spinel structure, which leads to well dispersed Cu nano particles. Furthermore, the inventors hypothesize that upon calcination at higher temperatures, e.g. at 600° C., well dispersed CuO nano particles will form, which similarly will lead to well dispersed Cu nano particles upon reduction (activation). Another advantage is that the zinc spinel formed after activation of the catalyst provides a higher and more stable surface area to disperse the Cu nano particles than zinc oxide does, leading to a higher stability compared to prior art catalysts. In fact, ZnAl₂O₄ seems to form smaller particles than ZnO, given the same calcination temperature.

As the skilled person will know, the aluminate ion of step ii. is only stable at high pH. Accordingly, it should be dissolved in a strongly basic solution such as an alkali hydroxide solution and/or an alkali carbonate solution. The Cu and Zn solution of i. is acidic. Both solutions are preferably aqueous solutions. The coprecipitation may be conducted by mixing equal volumes of i. and ii. and adjusting the pH to remain around a neutral pH. In the present context “neutral pH” is meant to refer to a pH in the range of from 6-9. The coprecipitation step a. may be conducted at a pH in the range of 6-12, such as in the range of 6-9, 7-9, 7.2-9, or 7.5-8.5.

In the method according to the present invention, the use of NaAlO₂ and similar aluminate salts seems to lead to the direct precipitation of a mixed Cu—Zn spinel phase, exemplified by the reaction

Cu²⁺+Zn²⁺+4AlO₂⁻=2(Cu_{0.5}Zn_{0.5})Al₂O₄

Without being bound by theory it is hypothesized that this is the key to obtaining the improved hydrogenation catalysts of the invention

According to another aspect of the present invention a process is provided for hydrogenating a carbonyl group of an organic carbonyl compound into its corresponding hydroxyl group, the process comprising contacting the organic carbonyl compound with a reduced form of the catalyst composition according to an aspect of the invention, in the presence of hydrogen to obtain an alcohol corresponding to said organic carbonyl compound.

According to an aspect of the present invention a use is provided of the catalyst according to the present invention, for hydrogenation of a feed comprising at least two of the carbonyl compounds selected from the group comprising formaldehyde, glycolaldehyde, glyoxal, pyruvic aldehyde and acetol.

The inventors found that all the advantages related to the catalyst according to the invention made it highly suitable for use in hydrogenation of biobased feedstocks and in particular of feedstocks derived from thermolytic fragmentation of sugars. In particular for industrial scale hydrogenation.

According to another aspect of the present invention a use is provided of alkali aluminate, such as potassium aluminate or sodium aluminate, for preparing a catalyst composition for hydrogenation reactions.

The inventors found that using alkali aluminate as aluminum source, dissolving it in a basic solution and coprecipitating it with an acidic solution comprising copper and zinc ions, provided an improved precursor which upon calcination at 250-900° C. provided catalyst compositions having much higher amounts of spinel phase than prior art Cu/Zn/Al catalysts. In particular, the inventors found that at lower calcination temperatures in the range of 250-550° C. most copper and zinc would be bound as a mixed spinel of the type CuₓZn₁₋ₓAl₂O₄. The spinel phase may make up as much as above 90% by weight of the catalyst composition as determined by XRD at the lower calcination temperatures in the range of 250-550° C. Without being bound by theory, the inventors hypothesize that an advantage of this is that Cu is retained in the spinel structure and only upon heating above 450° C. a phase transition takes place resulting in a fraction of the copper particles transforming into copper oxide. This somehow seems to result in a higher dispersion of the copper oxide compared to prior art catalyst compositions.

### FIGURES

FIG. 1 shows the correlation between the fraction of visible CuO per total amount of copper oxide present (Z) and calcination temperature (Tcalc) for oxidized forms of catalysts of the invention as well as for comparative catalysts H and I.

FIG. 2 shows the phase composition of catalyst D450 in its oxidized form vs temperature measured in steps of 50° C. A phase transition shows at close to 600° C., where a disordered spinel—the mixed Cu/Zn-spinel—transforms into CuO+ZnAl₂O₄. Below the transition temperature, there is almost no CuO visible by XRD (Example 4).

FIG. 3 shows the phase composition of catalyst E450 in its oxidized form vs temperature measured in steps of 50° C. A phase transition shows at close to 600° C., where a disordered spinel—the mixed Cu/Zn-spinel—transforms into CuO+ZnAl₂O₄. In this catalyst, a small amount of CuO is present also at low temperature (Example 8).

FIG. 4 shows the conversion of acetol into propylene glycol after 60 hours on stream by hydrogenation over catalysts according to the invention of the F series which have been calcined at various calcination temperatures (Tcalc) (Example 29).

FIG. 5 shows the BuOH yields at start of run (SOR) and end of run (EOR) for Catalyst A, Catalyst F450, Comparative Catalyst I and Comparative Catalyst K (Example 30).

FIG. 6 shows the stability, calculated as the BuOH yield at EOR relative to the BuOH yield at SOR for Catalyst A, Catalyst F450, Comparative Catalyst I and Comparative Catalyst K (Example 30).

FIG. 7 shows the BuOH yield per Wt % Cu for the three Cu catalysts; Catalyst A, Catalyst F450 and Comparative catalyst I (Example 30).

FIG. 8 shows a significant propane formation for the Ni catalyst (Comparative Catalyst K) (Example 30).

FIG. 9 shows the radial strength or side crush strength (SCS) for Catalyst A, Catalyst F450, Comparative Catalyst I and Comparative Catalyst J (Example 30).

FIG. 10 shows Side Crush Strength vs tablet density for various catalysts of the invention and of comparative catalysts.

FIG. 11 shows copper surface area, SA(Cu), vs Zn/Al molar ratio for four catalysts of the invention, all calcined at Tcalc=450° C., and furthermore, for two comparative catalysts, likewise with Tcalc=450° C. (Example 31).

FIG. 12 compares catalysts of the invention with similar Cu content (23±3 Wt % Cu) and Zn/Al=0.46±0.02 but with different calcination temperature (Tcalc).

FIG. 13. shows an exemplary XRD diffractogram of Catalyst E calcined at 450 (Example 8), 600 (Example 10) and 800° C. (Example 13), respectively.

FIG. 14 shows a visual inspection of to the left Comparative catalyst I calcined at 450° C.; and to the right Catalyst B calcined at 450° C.

### DETAILED DESCRIPTION

In the context of the present invention when referring to X-ray diffraction (XRD) this is meant to refer to XRD analysis yielding phase composition and lattice parameters, for example carried out based on powder X-ray diffraction measured in Bragg-Brentano geometry, with Cu Kα radiation, and analyzed using a full profile Rietveld analysis. Such analysis will indicate the size of the crystals in the powder analyzed. The larger the crystals of the materials, the more narrow the X-ray diffractogram peaks.

When referring to the content of metals present in the catalyst, such contents may be calculated by elemental analysis, such as by the ICP-OES method.

The copper surface area, SA(Cu), may be determined by surface titration of the catalyst in its reduced form with nitrous oxide; the so-called N₂O-RFC method as explained in S. Kuld et al. Angewandte Chemie 53 (2014), 5941-5945

Pore volumes (PV) may be determined by the mercury intrusion method. The mercury intrusion is conducted according to ASTM D4284.

Mechanical strength is measured as the Side Crush Strength (SCS) according to ASTM D4179-11.

Acid resistance may be determined by the acid resistance test involving boiling of pre-reduced and passivated catalyst in butyl benzoate/benzoic acid/water for 24 hours and then visually inspecting how much of the catalyst was intact, retaining its overall geometrical shape.

In the present context “catalyst precursor”, “catalytic precursor composition” “precursor” and “precursor composition” all refer to the composition obtained after coprecipitation and drying but before calcination.

In the present context “catalyst”, “composition for catalytic hydrogenation”, catalytic composition” and “catalyst composition” all refer to the composition after calcination. The catalyst is in its oxidized when in an oxidizing atmosphere, such as air or its reduced (active) form when in a reducing atmosphere, such as hydrogen gas. The reduced form is the form where the composition is considered catalytically active in hydrogenation reactions.

Catalytic Composition and Preparation Thereof

In an embodiment of the present invention, the catalysts do not contain Cr or Ni. In an embodiment according to the present invention the catalyst composition in its oxidized form comprises less than 0.01 Wt % Ni and/or less than 0.01 Wt % Cr. The inventive catalysts comprise, in their oxidized form, oxides of Cu, Zn and Al.

Said catalyst comprising Cu, Zn and Al and further being characterized, in its oxidized form, by
e) having a Cu content in the range of 12-38% by weight, such as in the range of 18-25% by weight, a Zn content in the range of 13-35%, such as in the range of 13-24% and an Al content in the range of 12-30%, such as in the range of 17-24%
f) having a molar ratio between Zn and Al in the interval 0.24-0.60, preferably in the interval 0.30-0.55, more preferably in the interval 0.35-0.50, most preferably in the interval 0.40-0.499 g) having a phase composition which, according to X-ray diffraction, includes a spinel phase and optionally a zinc oxide phase, the sum of which accounts for in the interval Q-100% by weight of all oxidic phases in the catalyst, where Q depends on a maximum calcination temperature (Tcalc) the catalyst has been exposed to in air for a period of in the interval 1-10 hours, so that
g1) if 250° C. Tcalc 550° C., then Q=80, such as Q=90 or such as Q=95 or such as Q=99
g2) if 550° C.≤Tcalc≤900° C., then Q=50, such as Q=60
h) having a percentage Z of XRD-visible CuO, defined as the percentage Wt % CuO according to XRD relative to the maximum possible Wt % CuO calculated from bulk elemental analysis (ICP or similar method), where Z depends on the maximum calcination temperature (Tcalc) the catalyst has been exposed to in air for a period of in the interval 1-10 hours, so that 0<Z<0.125*Tcalc, where the unit of Tcalc is ° C.

In an embodiment of the method according to the invention the aluminate salt may be provided as an alkali aluminate selected from the group consisting of lithium, sodium, potassium, rubidium and cesium. It is considered within the capabilities of the skilled person to identify suitable sources of Cu and Zn. Particularly suitable are nitrate salts of Cu and Zn. It is also considered within the capabilites of the skilled person to estimale the releative amounts of the Cu, Zn and aluminate sources required to achieve the desired relative amounts of Cu, Zn and Al.

By limiting the Zn/Al molar ratio to the range 0.24-0.60, the amount of free ZnO in the active catalyst is limited since most of the Zn is bound in a spinel structure which is much less reactive towards acids than ZnO and which furthermore has a higher and more stable surface area than ZnO, thus providing a better support to disperse the Cu nano particles formed upon activation of the catalyst. According to an embodiment of the present invention catalyst composition comprises in its oxidized form less than 15% by weight of ZnO, such as less than 13, 11, 9, 8, 7, 6, 5, 4, 3, 2, 1% by weight of ZnO. By calcination at a temperature of at least 250° C., such as between 350° C. and 700° C. or preferably between 550° C. and 700° C., a spinel phase is formed which has improved mechanical strength, improved thermal stability (less sintering) and improved tolerance towards e.g. carboxylic acids. Without being bound by theory, it is hypothesized that the high amount of spinel phase and the resulting minimal sintering provides a large surface area for the Cu crystals to disperse on. Furthermore, limiting the Cu content to no more than 38% helps to ensure a sufficient mechanical strength in the catalysts of the invention.

In an embodiment of the method according to the invention the calcination of step b) of the catalyst precursor composition is conducted at a temperature Tcalc in the range of from 250-450° C. to obtain an oxidized form of a composition for catalytic hydrogenation of an organic carbonyl compound, the composition comprising in its oxidized form at least 75% by weight, such as at least 80% of a spinel structure as determined by X-ray diffraction.

In an embodiment, the method according to the invention has the calcining of the catalyst precursor composition to be conducted at a temperature Tcalc in the range of from 450-900° C., such as from 550-750° C. to obtain an oxidized form of a composition for catalytic hydrogenation of organic carbonyl compounds, the composition comprising in its oxidized form at least 50% by weight, such as at least 60% of a spinel structure as determined by X-ray diffraction.

In an embodiment, the method according to the invention has a percentage Z of visible CuO in the range of from 20% to 100%, defined as the percentage by weight of CuO according to XRD relative to the maximum possible percentage by weight of CuO calculated from the amount of Cu present in the catalyst precursor composition of step a).

The catalyst of the invention and the catalyst utilized in the process according to the invention is further characterized by a low content of zinc oxide (ZnO) as determined by powder X-ray diffraction (XRD). Free zinc oxide is sensitive to acids which may be present in the surroundings. Thus, the catalyst may deteriorate or lose mechanical strength in the presence of acids, if any significant amount of zinc oxide is present during hydrogenation/use. The key to achieving this low ZnO content is twofold. Thus, the Zn/Al molar ratio is in the range 0.24-0.60, such as in the range 0.40-0.499, which allows for the formation of zinc spinel (ZnAl₂O₄) with a Zn/Al ratio of 0.50, and calcination in the interval 250-900° C., such as 350-700° C., 450-800° C. or 550-700° C. ensures a high degree of spinel formation. The high content of zinc spinel ZnAl₂O₄ and the limited Cu-content ensures a high mechanical strength.

The catalyst composition may be defined by (in the oxidized form of the catalyst) a Cu-content in the range 12-38 wt %, such as 15-30 wt %, or such as 17-28 wt %, or such as 20-27%, and by a Zn/Al molar ratio in the range 0.24-0.60, such as in the range 0.30-0.55, or such as in the range 0.30-0.50, or such as in the range 0.40-0.499, where the content of zinc (as elemental Zn) is in the range 13-35 wt % and the content of aluminum (as elemental Al) is in the range 15-30 wt %. According to an embodiment of the present invention the catalyst composition has a molar ratio of Zn:Al in the range of from 0.30-0.55, such as from 0.35-0.50, or from 0.40-0.499. According to an embodiment of the present invention the catalyst composition comprises in its oxidized form 15-38% by weight of Cu, such as 15-28% or 18-28% or 20-25% by weight of Cu. According to an embodiment of the present invention the catalyst composition comprises in its oxidized form 13-24% by weight of Zn, such as 15-25% by weight of Zn. According to an embodiment of the present invention the catalyst composition comprises in its oxidized form 17-24% by weight of Al. According to an embodiment of the present invention the catalyst composition comprises in its oxidized form at least 60% by weight, such as at least 70%, 75%, 80%, 85% or 90% by weight of a spinel structure as determined by X-ray diffraction. Within these ranges high-performing catalyst compositions are obtained, but the optimal combination of those features may vary to some extent depending on the hydrogenation reaction to be catalyzed and the demands to catalyst stability, mechanical strength and chemical inertness.

In an embodiment of the invention, said catalyst has been exposed to a temperature Tcalc of between 250-900° C., such as between 350-700° C., 450-700° C., 450-800° C., 550-800° C.

In an embodiment of the invention, said catalyst has been exposed to a calcination temperature Tcalc, in the range of 550-700° C.

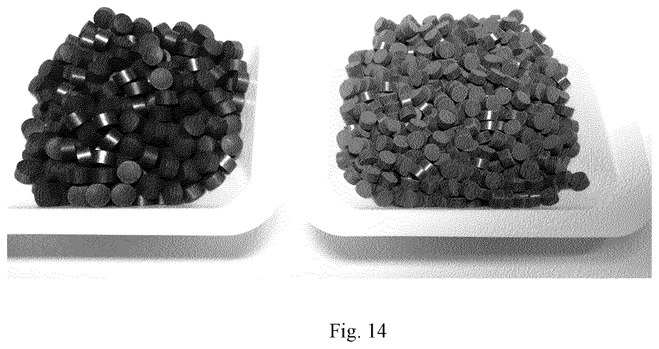

The oxidized form of the catalyst is the form obtained after calcination. The state of Cu depends on the calcination temperature, Tcalc, so that at low calcination temperature, typically in the interval 250-550° C., Cu forms a mixed spinel of the type CuₓZn₁₋ₓAl₂O₄ with only a small amount of the Cu being present as CuO. In this case the color of the catalyst (in its oxidized form) can be described as olive green (See FIG. 14 for color difference between catalysts of the invention compared to prior art). With increasing calcination temperature, the fraction of Cu present as CuO gradually increases, causing the catalyst to appear dark brown. The reduced form, also called the activated form, of the catalyst is the form obtained after reduction of the catalyst with a reducing agent, which is typically hydrogen, where Cu is present mainly or solely as elemental Cu.

Without being bound by theory, we believe that the phase transition that occurs in the oxidized forms of the catalysts of the invention when exposed to an O₂-containing atmosphere from low temperature (e.g. 450° C.) to high temperature (e.g. 650° C.) (during calcination) can be described as follows for a catalyst with a Zn/Al ratio of 0.50 and a Cu/Zn ratio of x:

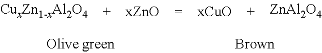

At low temperature, the ZnO present together with the mixed spinel phase is difficult to observe by XRD. This is probably due to a combination of low crystallinity of this phase and overlying diffraction peaks from the spinel phase.

It is important to note that the catalyst can be activated the same way no matter the calcination temperature and thus no matter the distribution of Cu(II) between the spinel phase and the cupric oxide (CuO) phase. Catalyst activation can be done e.g. by exposing the catalyst to a H₂-containing gas at a temperature in the interval 100-250° C., whereby the Cu(II) ions in the two phases, CuₓZn₁₋ₓAl₂O₄ and CuO, in both cases are transformed to elemental Cu.

On activation of the catalysts of the invention as e.g. by treatment with hydrogen at elevated temperature, elemental Cu is formed with high dispersion and thus high cupper surface area and accordingly high activity. Without being bound by theory, we believe that this high dispersion is a result of either small CuO particles formed in the above reaction by calcination at a temperature of 550-900° C., or is a result of reduction of the Cu(II) ions in the mixed spinel phase as in the catalysts calcined at 250-550° C. According to an embodiment of the present invention the catalyst composition has in its reduced form a copper metal surface area above 10 m²/g Cu, such as 10-30 or 10-20 m²/g Cu.

An important feature characterizing the oxidized form of the catalysts of the present invention is the percentage Z of XRD-visible CuO, defined as the percentage Wt % CuO according to XRD relative to the maximum possible Wt % CuO calculated from bulk elemental analysis (ICP or similar method):

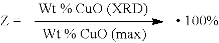

Thus, Z is a measure of how much of the Cu is present as CuO. If all Cu is present as CuO, Z is 100% while if no CuO is visible by XRD, Z is 0%.

Z depends on the maximum temperature the catalyst has been exposed to in atmospheric air for a period of in the interval of 1 to 10 hours (Tcalc), so that

0<Z<0.125·Tcalc

where the unit of Tcalc is ° C. This inequality is a characteristic of the catalysts of the present invention. FIG. 1 shows the value of Z for several catalysts of the present invention together with two comparative Cu/Zn/Al catalysts. It is evident that the two comparative catalysts have Z>95% (thus close to 100%) when calcined at 500° C., while the catalysts of the invention have Z<62.5% (0.125*500=62.5) at the same calcination temperature.

In an embodiment, the method according to the invention has a percentage Z of visible CuO in the range of from 0.1 to 23%, defined as the percentage by weight of CuO according to XRD relative to the maximum possible percentage by weight of CuO calculated from the amount of Cu present in the catalyst precursor composition of step a).

The phase composition of the oxidized form of the catalysts of the invention depends on the calcination temperature. If calcined at a temperature in the range 250-550° C., a spinel phase, (possibly including small amounts of ZnO), accounts for 80-100% by weight of the catalyst in oxidized form according to X-ray diffraction (XRD), while if calcined at a temperature in the range 550-900° C., the spinel phase accounts for 50-100% by weight of the catalyst in oxidized form.

According to an aspect of the present invention an oxidized form of a catalyst composition is provided which is obtainable by any of the embodiments of the method for preparing the catalyst or which is obtainable by any of the embodiments of the catalyst composition disclosed herein.

According to another aspect of the present invention a catalyst precursor composition is provided which is obtainable by step a. of the method according to the invention. The catalyst precursor composition is suitable for preparing a catalyst composition suitable for catalytic hydrogenation of an organic carbonyl compound in an industrial setting.

According to yet another aspect of the present invention a reduced form of a catalyst composition is provided which is obtainable by reducing the catalyst composition according to any of the embodiments of the catalyst composition disclosed herein.

Tabletting

In an embodiment of the invention, tablets of said catalyst in its oxidized form have a radial crush strength, SCS, of between 25 and 150 kp/cm, said tablets having a tablet density in the range of 1.45-2.35 g/cm³, such as in the range of 1.65-2.35 g/cm³.

In an embodiment of the invention, tablets of said catalyst in its freshly reduced form have a radial crush strength of between 10 and 75 kp/cm, said tablets having a tablet density in the interval 1.45-2.35 g/cm³, such as in the range 1.65-2.35 g/cm³.

Catalytic Hydrogenation

Thus, this invention provides a process for the catalytic hydrogenation of organic carbonyl compounds containing at least one functional group belonging to the group of aldehydes, ketones, esters and carboxylic acids, whereby said at least one functional group is converted to an alcohol by contacting said carbonyl compound with hydrogen and a hydrogenation catalyst according to the present invention at elevated temperature and pressure.

The following examples serve to illustrate the invention. Comparative examples are included.

### EXAMPLES

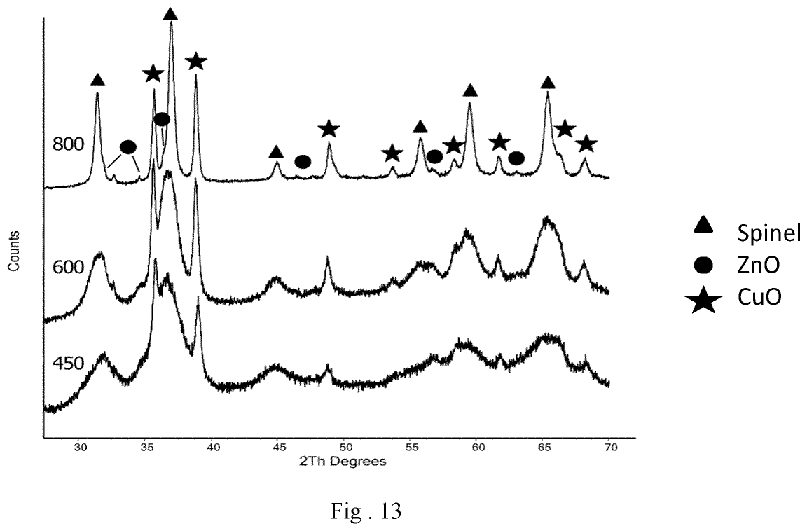

In the following examples it should be understood, that calcination is carried out by heating a sample of the catalyst, typically 1-10 gram, to the specified temperature for 4 hours. It should be noted that if the catalyst contains graphite, this will be combusted in air starting at around 550-600° C., contributing to increase the temperature in the catalyst. This effect is modest when handling small samples (1-10 gram) as can be observed by monitoring the temperature in the calcination crucible during calcination. When handling larger samples, excessive temperature rise must be prevented. Elemental analysis was carried out by the ICP-OES method. XRD analysis yielding phase composition and lattice parameters, was carried out based on powder X-ray diffraction measured in Bragg-Brentano geometry, with Cu Kα radiation, and analyzed using a full profile Rietveld analysis. See FIG. 13 for an exemplary XRD diffractogram of Catalyst E calcined at 450 (Example 8), 600 (Example 10) and 800° C. (Example 13), respectively.

### Example 1. Preparation of Catalyst A

Catalyst A was prepared by coprecipitation as follows. An aqueous solution containing 240 g Cu(NO₃)₂*2½H₂O and 333 g Zn(NO₃)₂*6H₂O was prepared and the volume was adjusted to 1 liter. Another solution containing 217 g NaAlO₂, 42 g NaOH and 38 g Na₂CO₃*10H₂O was prepared separately and the volume adjusted to 1 liter. Equal volumes of the two solutions were mixed at pH=8.0±0.2 using a third solution of Na₂CO₃*10H₂O to continuously adjust the pH. After precipitation, the product was ripened for 1 hour at 85° C. The product was filtered, washed several times with hot water and dried at 100° C. The powder was mixed with 4 wt % graphite and shaped in the form of cylindrical tablets, 4.5 mm diameter×3.5 mm height, which were finally calcined at 450° C. The composition of the catalyst was 18.5 wt % Cu, 20.6 wt % Zn and 20.2 wt % Al. Calculated as the oxides, this corresponds to a content of 23.2 wt % CuO, 25.6 wt % ZnO and 38.2 wt % Al₂O₃. The Zn/Al molar ratio based on the analysis was thus 0.42. According to powder X-ray diffraction (XRD) analysis, the sample contained (apart from the graphite) a spinel phase, possibly together with ZnO, while no CuO was visible. Measured as an average of 10 tablets, the tablet density was 1.88 g/cm³ and the radial crush strength was 49.3 kp/cm.

### Example 2. Preparation of Catalyst B

Catalyst B was prepared similarly to catalyst A but with an altered composition. Thus, the catalyst composition was found to be 23.5 wt % Cu, 19.8 wt % Zn and 18.6 wt % Al. Calculated as the oxides, this corresponds to a content of 29.4 wt % CuO, 24.6 wt % ZnO and 35.1 wt % Al₂O₃. The Zn/Al molar ratio based on the analysis was thus 0.44. According to XRD analysis, the sample contained (apart from graphite) a spinel phase, possibly together with ZnO, while no CuO was visible. Measured as an average of 10 tablets, the tablet density was 1.99 g/cm3 and the radial crush strength was 88.9 kp/cm.

### Example 3. Preparation of Catalyst C

Catalyst C was prepared similarly to catalyst A but with an altered composition. Thus, the catalyst composition was found to be 21.8 wt % Cu, 23.8 wt % Zn and 17.5 wt % Al. Calculated as the oxides, this corresponds to a content of 27.3 wt % CuO, 29.6 wt % ZnO and 33.1 wt % Al₂O₃. The Zn/Al molar ratio based on the analysis was thus 0.56. According to XRD analysis, the sample contained (apart from the graphite) a spinel phase, possibly together with ZnO, while no CuO was visible. By further heating to 900° C., the XRD phase composition was found to be 67% spinel, 4% ZnO and 29% CuO thus close to the theoretical amount of CuO of 27.3%.

### Example 4. Preparation of Catalyst D450

Catalyst D450 was prepared similarly to catalyst A but with an altered composition. Thus, the catalyst composition was found to be 23.7 wt % Cu, 19.2 wt % Zn and 20.2 wt % Al. Calculated as the oxides, this corresponds to a content of 29.7 wt % CuO, 23.9 wt % ZnO and 38.2 wt % Al₂O₃. The Zn/Al molar ratio based on the analysis was thus 0.39. The dried precursor was calcined at 450° C. According to XRD analysis, the sample contained (apart from the graphite) a spinel phase, possibly together with ZnO, while no CuO was visible.

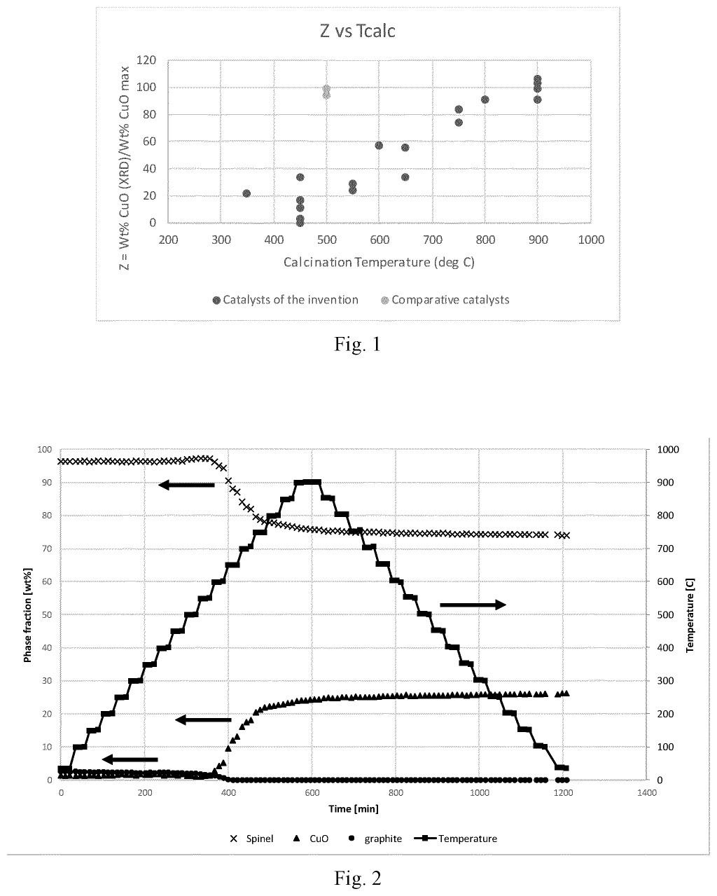

FIG. 2 shows the phase composition of this catalyst vs temperature measured in steps of 50° C. At the specific experimental conditions (the heating rate is particularly important), a phase transition shows at close to 600° C., where a disordered spinel—the mixed Cu/Zn-spinel—transforms into CuO+ZnAl₂O₄. Below the transition temperature, there is almost no CuO visible by XRD.

### Example 5. Preparation of Catalyst D550

Catalyst D550 was obtained from the dried precursor to Catalyst D450 by calcination at 550° C. According to XRD analysis, the sample contained (apart from the graphite) a spinel phase, possibly together with ZnO, while no CuO was visible. Prolonging the calcination to 50 hours at 550° C. caused a change in XRD phase composition, which at that point was found to be 92% spinel and 8% CuO.

### Example 6. Preparation of Catalyst D650

Catalyst D650 was obtained from the dried precursor to Catalyst D450 by calcination at 650° C. According to XRD analysis, the sample contained 90% of a spinel phase and 10% CuO. Prolonging the calcination to 50 hours at 650° C. caused a change in XRD phase composition, which at that point was found to be 82% spinel and 18% CuO.

### Example 7. Preparation of Catalyst D750

Catalyst D750 was obtained from the dried precursor to Catalyst D450 by calcination at 750° C. According to XRD analysis, the sample contained 79% of a spinel phase and 21% CuO. Prolonging the calcination to 50 hours at 750° C. caused only a slight change in XRD phase composition, which at that point was found to be 78% spinel and 22% CuO.

By further heating to 900° C., the XRD phase composition was found to be 73% spinel and 27% CuO thus approaching the theoretical amount of CuO of 29.7% as given in Example 4.

### Example 8. Preparation of Catalyst E450

Catalyst E450 was prepared similarly to catalyst A but with an altered composition. Furthermore, the catalyst powder was not tablettized and was therefore not mixed with graphite. The catalyst composition was found to be 20.1 wt % Cu, 21.4 wt % Zn and 19.8 wt % Al. Calculated as the oxides, this corresponds to a content of 25.2 wt % CuO, 26.6 wt % ZnO and 37.4 wt % Al₂O₃. The Zn/Al molar ratio based on the analysis was thus 0.45. According to XRD analysis, the sample contained 91% of a spinel phase, possibly together with ZnO, and 9% CuO.

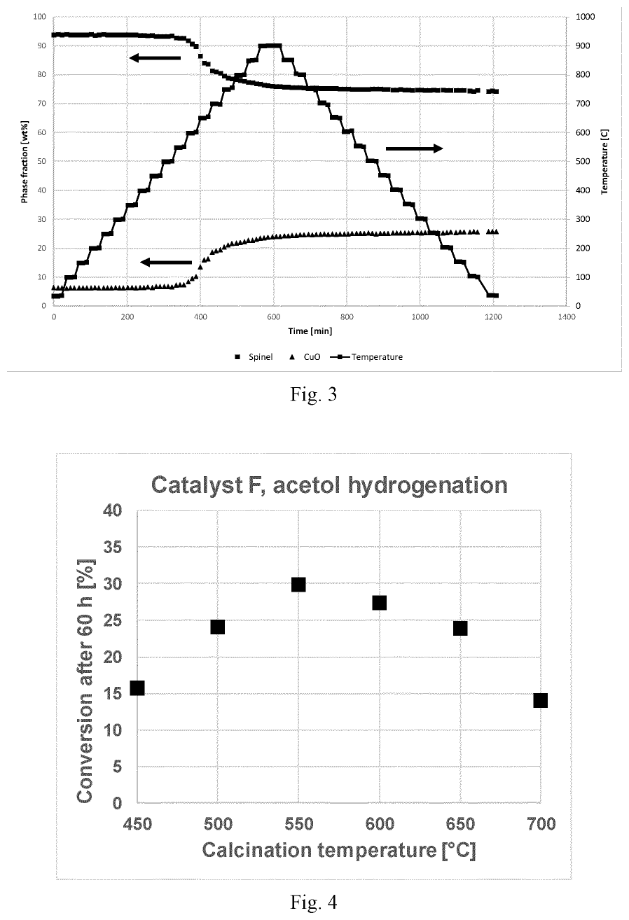

FIG. 3 shows the phase composition of this catalyst vs temperature measured in steps of 50° C. At the specific experimental conditions (the heating rate is particularly important), a phase transition shows at close to 600° C., where a disordered spinel—the mixed Cu/Zn-spinel—transforms into CuO+ZnAl₂O₄. In this catalyst, a small amount of CuO is present also at low temperature.

### Example 9. Preparation of Catalyst E550

Catalyst E550 was obtained from the dried precursor to Catalyst E450 by calcination at 550° C. According to XRD analysis, the sample contained 95% of a spinel phase, possibly together with ZnO, and 5% CuO. Prolonging the calcination to 50 hours at 550° C. caused a change in XRD phase composition, which at that point was found to be 92% spinel and 8% CuO.

### Example 10. Preparation of Catalyst E600

Catalyst E600 was obtained from the dried precursor to Catalyst E450 by calcination at 600° C. According to XRD analysis, the sample contained 83% of a spinel phase, 3% ZnO and 14% CuO.

### Example 11. Preparation of Catalyst E650

Catalyst E650 was obtained from the dried precursor to Catalyst E450 by calcination at 650° C. According to XRD analysis, the sample contained 86% of a spinel phase and 14% CuO. Prolonging the calcination to 50 hours at 650° C. caused a change in XRD phase composition, which at that point was found to be 81% spinel and 19% CuO.

### Example 12. Preparation of Catalyst E750

Catalyst E750 was obtained from the dried precursor to Catalyst E450 by calcination at 750° C. According to XRD analysis, the sample contained 79% of a spinel phase and 21% CuO. Prolonging the calcination to 50 hours at 750° C. caused a change in XRD phase composition, which at that point was found to be 78% spinel and 22% CuO.

By further heating to 900° C., the XRD phase composition was found to be 75% spinel and 25% CuO thus approaching the theoretical amount of CuO of 25.2% as given in Example 8.

### Example 13. Preparation of Catalyst E800

Catalyst E800 was obtained from the dried precursor to Catalyst E450 by calcination at 800° C. According to XRD analysis, the sample contained 75% of a spinel phase, 2% ZnO and 23% CuO.

### Example 14. Preparation of Catalyst F350

Catalyst F350 was prepared similarly to catalyst A but with an altered composition and with a calcination temperature of 350° C. According to XRD analysis, the sample contained (apart from graphite) 94% of a spinel phase, possibly together with ZnO, and 6% CuO. The color of the catalyst is olive green.

### Example 15. Preparation of Catalyst F450

Catalyst F450 was obtained from the dried precursor to Catalyst F350 by calcination at 450° C. The catalyst composition was found to be 24.4 wt % Cu, 19.7 wt % Zn and 17.0 wt % Al. Calculated as the oxides, this corresponds to a content of 30.5 wt % CuO, 24.5 wt % ZnO and 32.1 wt % Al₂O₃. The Zn/Al molar ratio based on the analysis was thus 0.48. According to XRD analysis, the sample contained (apart from graphite) 94% of a spinel phase, possibly together with ZnO, and 6% CuO. Measured as an average of 10 tablets, the tablet density was 1.94 g/cm³ and the radial crush strength was 53.3 kp/cm.

### Example 16. Preparation of Catalyst F500

Catalyst F500 was obtained from the dried precursor to Catalyst F350 by calcination at 500° C. According to XRD analysis, the sample contained (apart from graphite) 87.4% of a spinel phase, possibly together with ZnO, and 12.6% CuO.

### Example 17. Preparation of Catalyst F550

Catalyst F550 was obtained from the dried precursor to Catalyst F350 by calcination at 550° C. According to XRD analysis, the sample contained (apart from graphite) 86.7% of a spinel phase, possibly together with ZnO, and 13.3% CuO.

### Example 18. Preparation of Catalyst F600

Catalyst F600 was obtained from the dried precursor to Catalyst F350 by calcination at 600° C. According to XRD analysis, the sample contained (apart from graphite) 84.9% of a spinel phase, possibly together with ZnO, and 15.1% CuO. The color of the catalyst is dark brown.

### Example 19. Preparation of Catalyst F650

Catalyst F650 was obtained from the dried precursor to Catalyst F350 by calcination at 650° C. According to XRD analysis, the sample contained (apart from graphite) 77% of a spinel phase, possibly together with ZnO, and 23% CuO.

### Example 20. Preparation of Catalyst F700

Catalyst F700 was obtained from the dried precursor to Catalyst F350 by calcination at 700° C. According to XRD analysis, the sample contained (apart from graphite) 72.2% of a spinel phase, possibly together with ZnO, and 27.8% CuO.

### Example 21. Preparation of Catalyst G

Catalyst G was prepared similarly to catalyst A but with an altered composition. Furthermore, the catalyst powder was not tablettized and was therefore not mixed with graphite. The catalyst composition was found to be 22.4 wt % Cu, 13.8 wt % Zn and 23.4 wt % Al. Calculated as the oxides, this corresponds to a content of 28.0 wt % CuO, 17.2 wt % ZnO and 44.2 wt % Al₂O₃. The Zn/Al molar ratio based on the analysis was thus 0.24. According to XRD analysis, the sample contained 99% of a spinel phase, possibly together with ZnO, and 1% CuO. By further heating to 900° C., the XRD phase composition was found to be 71% spinel and 29% CuO thus close to the theoretical amount of CuO of 28%.

### Comparative Example 22. Preparation of Catalyst H

Catalyst H was prepared similarly to catalyst A but with a different composition. Furthermore, the calcination temperature was 350° C. The catalyst composition was found to be 41.0 wt % Cu, 22.2 wt % Zn and 5.5 wt % Al. Calculated as the oxides, this corresponds to a content of 51.3 wt % CuO, 27.6 wt % ZnO and 10.4 wt % Al₂O₃. The Zn/Al molar ratio based on the analysis was thus 1.67. Measured as an average of 10 tablets, the tablet density was 1.89 g/cm³ and the radial crush strength was 16.5 kp/cm. For analysis, a sample of Catalyst H was calcined at 500° C. and analyzed by ICP and XRD giving a Z-value of 94% (FIG. 1).

### Comparative Example 23. Preparation of Catalyst I

Catalyst I was prepared similarly to catalyst A but with a different composition. Furthermore, the calcination temperature was 350° C. The catalyst composition was found to be 45.6 wt % Cu, 20.0 wt % Zn and 4.6 wt % Al. Calculated as the oxides, this corresponds to a content of 57.1 wt % CuO, 24.9 wt % ZnO and 8.7 wt % Al₂O₃. The Zn/Al molar ratio based on the analysis was thus 1.79. Measured as an average of 10 tablets, the tablet density was 1.97 g/cm³ and the radial crush strength was 29.4 kp/cm. Another batch of tablets had a tablet density of 1.90 and a radial crush strength of 45 kp/cm. For analysis, a sample of Catalyst I was calcined at 500° C. and analyzed by ICP and XRD giving a Z-value of 99% (see FIG. 1).

### Comparative Example 24. Preparation of Catalyst J

Catalyst J is a copper chromite purchased from Merck as a powder. The powder was mixed with 4% graphite and compressed to 4.5 mm diameter×3.5 mm height cylindrical tablets. The catalyst composition was found to be 37.1 wt % Cu and 29.5 wt % Cr, which roughly corresponds to the stoichiometry CuO*CuCr₂O₄. Calculated as the oxides, this corresponds to a content of 46.4 wt % CuO and 43.1 wt % Cr₂O₃. Measured as an average of 10 tablets, the tablet density was 2.76 g/cm³ and the radial crush strength was 16.6 kp/cm.

### Comparative Example 25. Preparation of Catalyst K

Catalyst K is a Ni catalyst made by impregnation of an alumina support. The powder was mixed with 4% graphite and compressed to 4.5 mm diameter×3.5 mm height cylindrical tablets. The catalyst was found to contain 14.5 Wt % Ni.

### Example 26. Acid Resistance Test of Catalyst A

25 g of Catalyst A was pre-reduced by heating to 220° C. and treatment with 5% hydrogen in nitrogen at 50 Nl/h for four hours. The catalyst was cooled to room temperature and passivated by treatment with 1% oxygen in nitrogen at 50 Nl/h for two hours. This passivation procedure causes a surface oxidation of the copper particles. Thus, X-ray powder diffraction reveals that most of the copper is present as metallic Cu while only a minor fraction is present as Cu₂O and very little as CuO. For the acid resistance test, 5 grams of benzoic acid and 1 gram of water were dissolved in 94 grams of butyl benzoate (boiling point=250° C.). 5 g of pre-reduced and passivated Catalyst A in the shape of 4.5×3.5 mm tablets was added. The suspension was heated to reflux for 24 hours. The liquid was decanted off and the tablets were inspected. It was found that most of the tablets were intact and almost no powder was observed.

### Example 27. Acid Resistance Test of Catalyst B

25 g of Catalyst B was reduced and passivated as described in Example 26. The acid resistance test (boiling in butyl benzoate/benzoic acid/water for 24 hours) was carried out as in Example 26. The liquid was decanted off and the tablets were inspected. The majority of the tablets were intact and the appearance was similar to that of Catalyst A.

### Comparative Example 28. Acid Resistance Test of Catalyst H

25 g of Catalyst H was reduced and passivated as described in Example 4. The acid resistance test (boiling in butyl benzoate/benzoic acid/water for 24 hours) was carried out as in Example 4 with 5 g of catalyst. The catalyst was found to have deteriorated completely. Thus, no tablets were identified. Instead, a dark brown mud was found in the bottom of the flask.

### Example 29. Test of Catalysts for Hydrogenation of Acetol to Propylene Glycol

These tests were carried out separately with Catalyst F450, F500, F550, F600, F650 and F700. 50 mg catalyst was mixed with 6 g of SiC, both in the sieve fraction 0.15-0.30 mm. The mixture was loaded into a cylindrical reactor with inner diameter 5.0 mm. The catalyst was reduced with dilute hydrogen as described in Example 30. The reactor was heated to 230° C. Liquid feed (acetol and water) was evaporated and mixed with gaseous feed (H₂ and CO₂) to give a feed composition of 2.5 mol % acetol, 10.3 mol % H₂O, 67.1 mol % H₂ and 20.1 mol % CO₂. The reaction was carried out at P=0.3 MPa and T=230° C. at a total feed flow of 35.8 Nl/h. Results in terms of acetol conversion after 60 hours on stream are shown in FIG. 4. While all the catalysts are active for hydrogenation of acetol to propylene glycol, there is clearly an optimum in calcination temperature at 550° C. Acetol is hydroxyacetone.

### Example 30. Test of Catalysts for Hydrogenation of Butyraldehyde to n-Butanol (BuOH)

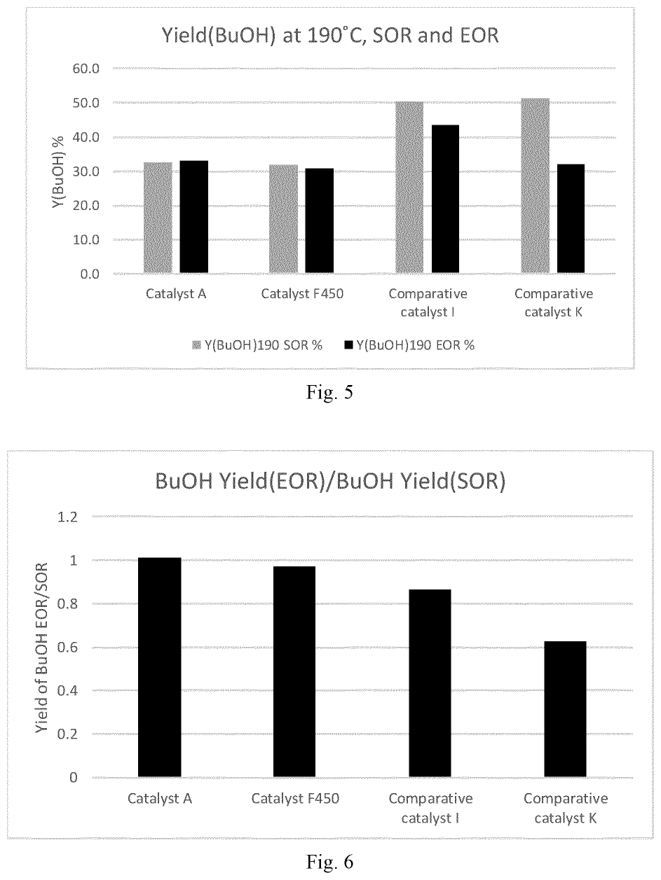

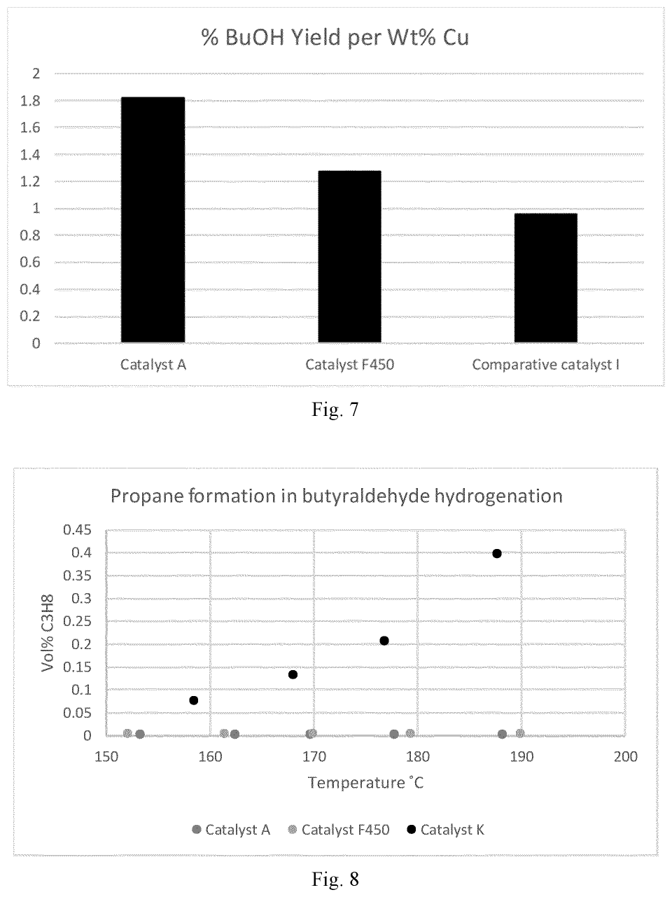

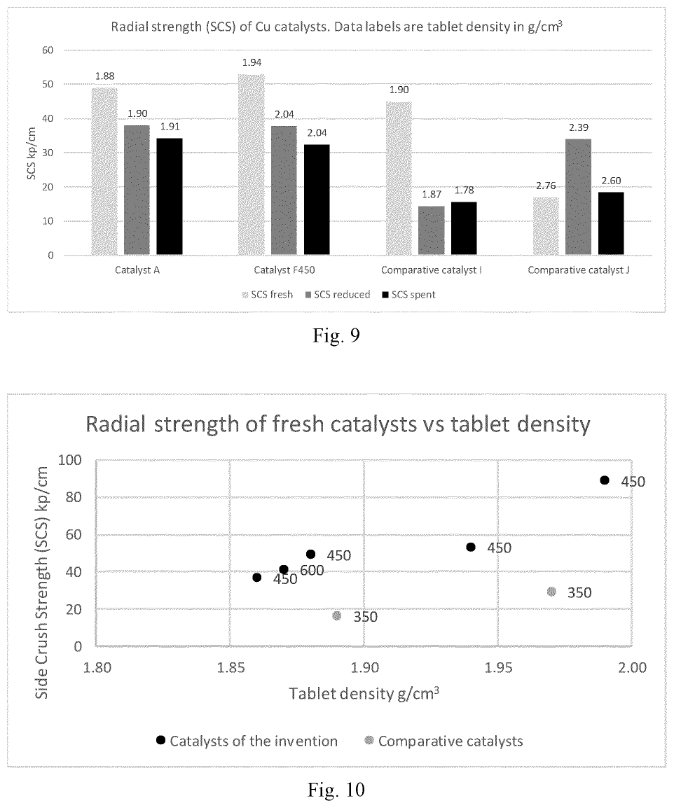

A 6.2 mm cylindrical, copper-lined reactor was loaded in Single Pellet String fashion with 6 catalyst tablets, each tablet separated from its neighbors by 4 spheres of dead-burned alumina. Prior to test, the catalyst was reduced with dilute hydrogen (3.0% H₂ in N₂) at 150-220° C. (2° C. per minute, hold at 220° C. for 2 hours). The Ni catalyst was reduced at 400° C. The tests were carried out at a pressure of 10 barg with a flow rate of 41.9 g/h butyraldehyde (13 Nl/h) and 75 Nl/h H₂. Butyraldehyde was evaporated and mixed with hydrogen before entering the reactor. The amount of loaded catalyst was 0.68 cm³ resulting in a GHSV of 129412 Nl/h. These experiments allowed for comparison of hydrogenation activity of butyraldehyde between the various catalysts. Four catalysts (Catalyst A, Catalyst F450, Comparative Catalyst I and Comparative Catalyst K) were tested separately under these conditions over a 50-hours period at the temperatures 190, 180, 170, 160, 150 and again at 190° C. The exit gases were analyzed by gas chromatography (GC). The non-condensable part of the exit gas was analyzed by an on-line GC and satisfying carbon mass balances were obtained for all measurements (C(ex)/C(in)=1.00±0.03). The BuOH yield was calculated based on all the GC analysis. The high GHSV ensured a butyraldehyde conversion in the range 13.5-51.3% for all catalysts within the entire temperature range. The BuOH selectivity based on the condensable part of the exit gas was in the range of 99.97-99.99% for all catalysts over the entire temperature range. However, while only H₂ was observed in the non-condensable part of the exit gas for the Cu-based catalysts, propane and CO were observed with the Ni-based catalysts in increasing amounts with increasing temperature. The BuOH yield at start of run (SOR) and end of run (EOR) for each of the four catalysts is shown in FIG. 5. While the BuOH yield is lower for the two catalysts of the invention than for the two comparative catalysts, the stability, calculated as the BuOH yield at EOR relative to the BuOH yield at SOR, is much better for the catalysts of the invention, as shown in FIG. 6. Furthermore, between the three Cu catalysts (Catalyst A, Catalyst F450 and Comparative catalyst I), the BuOH yield per Wt % Cu is significantly higher for the two catalysts of the invention than for the comparative catalyst, FIG. 7. As for the Ni catalyst, Comparative Catalyst K, significant propane formation was observed, probably by decarbonylation of butyraldehyde, see FIG. 8. Finally, radial strength or side crush strength (SCS) was measured for Catalyst A, Catalyst F450, Comparative Catalyst I and Comparative Catalyst J—see FIG. 9. In all cases the SCS was measured on fresh, reduced and spent catalysts. Clearly, the catalysts of the present invention have a much higher mechanical strength than the two comparative catalysts. FIG. 10 shows SCS vs tablet density.

### Example 31. Copper Surface Areas

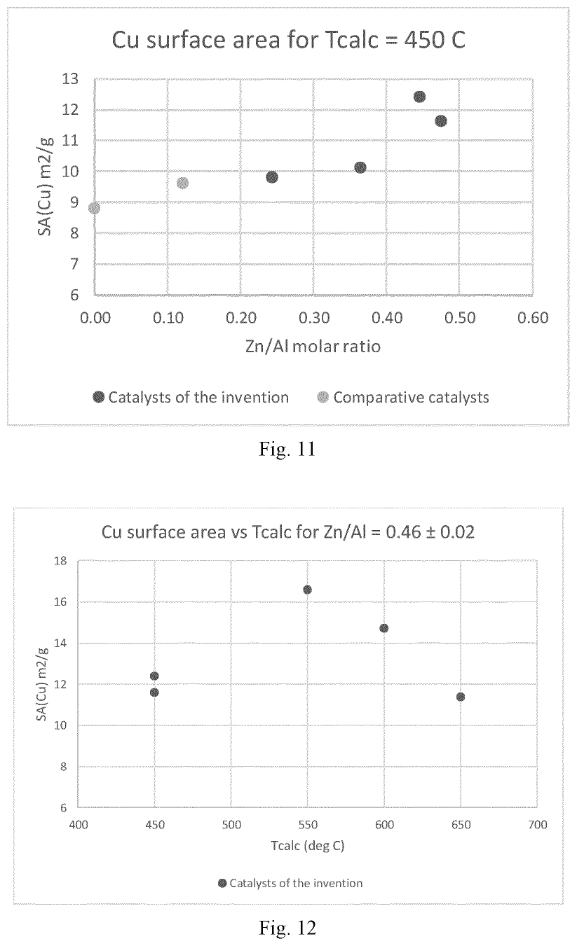

Some of the catalysts of the invention were studied by measurement of the copper surface area, SA(Cu), by surface titration with nitrous oxide; the so-called N₂O-RFC method as explained in S. Kuld et al. Angewandte Chemie 53 (2014), 5941-5945 (Supporting Information). 500 mg catalyst in sieve fraction 150-300 um was loaded into a U-type quartz reactor with an inner diameter of 4.0 mm and the system was flushed with helium. The catalyst was reduced in 1% H₂ in N₂ from room temperature to 175° C. at a rate of 1 K/min and a hold time at 175° C. for 2 hours. Reduction continued, heating at 1 K/min from 175° C. to 250° C. and a hold time of 10 minutes. Reduction gas was then switched to pure hydrogen and a hold time at 250° C. of 2 hours. The temperature was adjusted to 210° C. and maintained in a flow of He for 40 min and then cooled to 50° C. The reactor was then closed off and isolated in He atmosphere at 50° C. The system with the reactor bypassed was flushed with 1% N₂O in N₂, first 5 min at a flow of 50 Nml/min and then 5 minutes at a flow of 12 Nml/min. The reactor was opened, and the catalyst surface was titrated at 50° C. for 35 minutes in the 1% N₂O at a flow of 12 Nml/min and the consumed N₂O in this step was used to calculate the Cu surface area. All gas flows were at a rate of 100 Nml/min unless stated otherwise. The copper surface area was calculated as SA(Cu)=0.081905 m² Cu/μmol N₂O. The copper surface area (m² Cu area per gram of catalyst) very often correlates with catalytic activity, since it is a measure of the number of active sites. This is not strictly correct since most Cu catalysts are structure sensitive, and also since the support or part of the support may impact the Cu sites or the catalytic cycle. Nevertheless, those who are skilled in the art would expect that the most active catalysts are those with the highest SA(Cu). This is indeed what we observe, at least qualitatively. Other factors, such as the porosity of the catalyst, may also influence the observed activity and other catalyst performance parameters to some extent. FIG. 11 shows SA(Cu) vs Zn/Al molar ratio for four catalysts of the invention, all calcined at Tcalc=450° C., and furthermore, for two comparative catalysts, likewise with Tcalc=450° C. All catalysts were prepared as described in Example 1, but with different compositions, in particular with respect to the Zn/Al molar ratio. The Cu content in all six catalysts varied only moderately, from 20.1-27.3 Wt %. It is clear, that SA(Cu) increases with the Zn/Al ratio, especially for the two catalysts with a Zn/Al ratio in the preferred interval of 0.40-0.50. We consider the catalyst with a Zn/Al ratio of 0.24 as a catalyst of the invention, since SA(Cu) also depends on calcination temperature, and since this catalyst belongs to the group of catalysts which benefit from a higher calcination temperature while the two comparative catalysts do not. The effect of calcination temperature is shown in FIG. 12. Here, catalysts of the invention with similar Cu content (23±3 Wt % Cu) and Zn/Al=0.46±0.02 but with different calcination temperature, Tcalc, are compared. Clearly, SA(Cu) is largest for a calcination temperature of around 550° C.

### Example 32. Catalyst Pore Volumes

Catalyst pore volumes (PV) were measured by mercury intrusion for selected catalysts of the invention. A higher PV is beneficial if the catalytic reaction is mass transfer limited. The pore volume and the porosity will depend on the tablet density. For typical tablet densities, which are in the range 1.7-2.1 g/cm³, the pore volume (PV) is in the range 150-350 ml/kg and the porosity is in the range 35-65%. For tablets with a tablet density in the range 1.8-2.0 g/cm³, we find PV in the range 200-300 ml/kg and porosity in the range 40-60%. We find that the highest PV and porosity is achieved by calcination at around 600° C.; see Table 2.

Tables 1, 2 and 3 gather examples of catalysts of the invention and comparative catalysts. All characterization data are obtained from catalysts in their oxidized form except for the copper surface area and the acid resistance which was determined on the reduced catalyst compositions.

TABLE 1
Catalyst characterization.

| | | | | | | | | | | | Compo- | | |
|---|---|---|---|---|---|---|---|---|---|---|---|---|---|
| | | | | | | | | | | | sition | | |
| | | | | | | | | | | | Metal | | |
| | | Tcal | | | | | | Wt % oxides (XRD) | | | Mole % | | Z |
| | | C | Wt % oxides (ICP) | | | Tablet | | | Wt | Wt | % Cu/ | Zn/Al | % |
| Example | | deg | Wt % | Wt % | Wt % | density | SCS | Wt % | % | % | % Zn/ | molar | XRD/ |
| # | Catalyst | C. | CuO | ZnO | Al2O3 | g/cm3 | kp/cm | Spinel | ZnO | CuO | % Al | ratio | ICP |
| 1 | A | 450 | 23.16 | 25.64 | 38.17 | 1.88 | 49.3 | 83.7 | 13.4 | 2.9 | 20/24/55 | 0.42 | 11.3 |
| 2 | B | 450 | 29.42 | 24.64 | 35.15 | 1.99 | 88.9 | 100 | *** | 0.0 | 27/22/51 | 0.44 | 0.0 |
| 3 | C | 450 | 27.29 | 29.62 | 33.07 | — | — | 100 | *** | 0.0 | 25/27/48 | 0.56 | 0.0 |
| 3* | C900 | 900 | 27.29** | — | — | — | — | 67 | 4.0 | 29.0 | — | — | 106.3 |
| 4 | D450 | 450 | 29.67 | 23.90 | 38.17 | 1.93 | — | 100 | *** | 0.0 | 26/21/53 | 0.39 | 0.0 |
| 5 | D550 | 550 | 29.67** | — | — | — | — | 92 | *** | 8.0 | — | — | 24.3 |
| 6 | D650 | 650 | 29.67** | — | — | — | — | 90 | *** | 10.0 | — | — | 33.7 |
| 7 | D750 | 750 | 29.67** | — | — | — | — | 78 | *** | 22.0 | — | — | 74.2 |
| 7* | D900 | 900 | 29.67** | — | — | — | — | 73 | *** | 27.0 | — | — | 91.0 |
| 8 | E450 | 450 | 25.16 | 26.64 | 37.41 | 1.86 | 36.7 | 86.8 | 3.8 | 9.4 | 23/24/53 | 0.45 | 33.6 |
| 9 | E550 | 550 | 25.16** | — | — | — | — | 92 | *** | 8.0 | — | — | 28.6 |
| 10 | E600 | 600 | 25.16** | — | — | 1.87 | 41.4 | 82.6 | 3.0 | 14.4 | — | — | 57.2 |
| 11 | E650 | 650 | 25.16** | — | — | — | — | 86 | *** | 14.0 | — | — | 55.6 |
| 12 | E750 | 750 | 25.16** | — | — | — | — | 79 | *** | 21.0 | — | — | 83.5 |
| 13 | E800 | 800 | 25.16** | — | — | 2.00 | 34.9 | 75.4 | 1.8 | 22.8 | — | — | 90.6 |
| 13* | E900 | 900 | 25.16** | — | — | — | — | 75 | *** | 25.0 | — | — | 99.4 |
| 14 | F350 | 350 | 25.16** | — | — | — | — | 94 | *** | 6.0 | — | — | 21.5 |
| 15 | F450 | 450 | 30.54 | 24.52 | 32.12 | 1.94 | 53.3 | 88 | 6.3 | 5.6 | 29/23/48 | 0.48 | 16.5 |
| 16 | F500 | 500 | 30.54** | — | — | — | — | 87.4 | *** | 12.6 | — | — | 37.1 |
| 17 | F550 | 550 | 30.54** | — | — | — | — | 86.7 | *** | 13.3 | — | — | 39.2 |
| 18 | F600 | 600 | 30.54** | — | — | — | — | 84.9 | *** | 15.1 | — | — | 49.4 |
| 19 | F650 | 650 | 30.54** | — | — | — | — | 77 | *** | 23.0 | — | — | 75.3 |
| 20 | F700 | 700 | 30.54** | — | — | — | — | 72.2 | *** | 27.8 | — | — | 91.0 |
| 21 | G | 450 | 28.04 | 17.18 | 44.22 | — | — | 95 | 4.0 | 1.0 | 25/15/61 | 0.24 | 3.2 |
| 21* | G900 | 900 | 28.04** | — | — | — | — | 71 | *** | 29.0 | — | — | 103.4 |
| 22 | H | 500 | 51.32 | 29.50 | 11.47 | — | — | 15.7 | 30.5 | 53.8 | 52/29/18 | 1.61 | 94.3 |
| 23 | I | 500 | 59.71 | 23.90 | 9.49 | — | — | 13.1 | 21.2 | 65.7 | 61/24/15 | 1.58 | 99.0 |
| 24 | J | 350 | 46.44 | 0.00 | 0.00 | 2.76 | 16.6 | 75.7 | 0.0 | 24.3 | — | — | 47.1 |

X*refers to example X but with some additional information.
**Content of CuO assumed the same as in the sample calcined at 450° C.
***No separate ZnO phase was detected by XRD.
″—″ means that the parameter has not been measured.

The sum of Wt % oxides (CuO+ZnO+Al₂O₃) is less than 100% (in the range 87-93%) due to residual water and to the added graphite lubricant.

TABLE 2
Catalyst Compositions by elemental analysis (ICP).

| | | | | | | Compo- | |
|---|---|---|---|---|---|---|---|
| | | | | | | sition | |
| | | | | | | Metal | |
| | | Tcalc | Wt % metals (ICP) | | | Mole % | Zn/Al |
| | | deg | Wt % | Wt % | Wt % | % Cu/ | molar |
| Example# | Catalyst | C. | Cu | Zn | Al | % Zn/% Al | ratio |
| 1 | A | 450 | 18.5 | 20.6 | 20.2 | 20/24/55 | 0.42 |
| 2 | B | 450 | 23.5 | 19.8 | 18.6 | 27/22/51 | 0.44 |
| 3 | C | 450 | 21.8 | 23.8 | 17.5 | 25/27/48 | 0.56 |
| 4 | D450 | 450 | 23.7 | 19.20 | 20.2 | 26/21/53 | 0.39 |
| 8 | E450 | 450 | 20.1 | 21.40 | 19.8 | 23/24/53 | 0.45 |
| 15 | F450 | 450 | 24.4 | 19.70 | 17 | 29/23/48 | 0.48 |
| 21 | G | 450 | 22.4 | 13.80 | 23.4 | 25/15/61 | 0.24 |
| 22 | H | 500 | 41 | 23.7 | 6.07 | 52/29/18 | 1.61 |
| 23 | I | 500 | 47.7 | 19.2 | 5.02 | 61/24/15 | 1.58 |
| 24 | J | 350 | 37.1 | 0 | 0 | — | — |

TABLE 3
Pore volume and porosity for selected catalysts.

| | | | Tablet | | |
|---|---|---|---|---|---|
| | | Tcalc | density | PV | Porosity |
| Example# | Catalyst | deg C. | g/cm3 | ml/kg | % |
| 1 | A | 450 | 1.88 | 241 | 45 |
| 2 | B | 450 | 1.99 | 214 | 43 |
| 4 | D450 | 450 | 1.93 | 256 | 45 |
| 8 | E450 | 450 | 1.86 | 255 | 50 |
| 10 | E600 | 600 | 1.87 | 278 | 56 |
| 13 | E800 | 800 | 2.00 | 248 | 53 |
| 15 | F450 | 450 | 1.94 | 244 | 45 |

Table 1 shows that catalysts of the present invention contain a spinel phase as the major phase according to XRD. Thus, for all the examples of the invention illustrating calcination temperatures in the range 350-900° C., the spinel content according to XRD is from 67-100%. The content of CuO according to ICP is in the range 23-31.5 Wt % in the examples, corresponding to 18-25 Wt % Cu. The invention includes catalysts with even higher Cu content, up to 38 Wt %. Even in that case, the spinel phase would make up at least 50% of the catalyst. The examples include catalysts with a Zn/Al molar ratio from 0.24 to 0.56. Table 1 includes calculated values of Z. This parameter is simply the ratio between Wt % CuO as observed by XRD to the theoretical or maximum Wt % CuO as calculated from ICP elemental analysis. In other words, the value of Z expresses how much of the Cu is present as a distinct CuO phase. The value of Z depends very much on the calcination temperature as shown in FIG. 1, and in general covers the entire range from 0-100%. The correlation with temperature is so that there is an upper limit for Z which depends on temperature so that 0<Z<0.125*Tcalc, where the unit of Tcalc is ° C.

Table 1 also lists examples of mechanical strength in terms of SCS. This is further addressed in FIG. 10, showing the very high strength of the catalyst of the invention.

Table 2 shows the elemental composition for selected catalysts. Catalysts of the invention have a Cu content of in the range 12-38% by weight, preferably in the range 18-25% by weight, a Zn content of in the range 13-35%, preferably in the range 13-24% and an Al content of in the range 12-30%, preferably in the range 17-24%.

Table 3 shows pore volume (PV) and porosity for selected catalysts of the invention. By comparison of examples 8, 10 and 13, it is seen that there is an optimum in porosity for a calcination temperature of 600° C.

### Embodiments

Embodiment 1. Process for the catalytic hydrogenation in gas phase or in liquid phase of organic carbonyl compounds containing at least one functional group belonging to the group of aldehydes, ketones, esters and carboxylic acids, whereby said at least one functional group is converted to an alcohol by contacting said carbonyl compound with hydrogen and a hydrogenation catalyst at elevated temperature and pressure, said catalyst comprising Cu, Zn and Al and further being characterized, in its fully oxidized form, by
e) having a Cu content of in the range 12-38% by weight, such as in the range 18-25% by weight, a Zn content of in the range 13-35%, such as in the range 13-24% and an Al content of in the range 12-30%, such as in the range 17-24%
f) having a molar ratio between Zn and Al in the interval 0.24-0.60, preferably in the interval 0.30-0.55, more preferably in the interval 0.35-0.50, most preferably in the interval 0.40-0.499
g) having a phase composition which, according to X-ray diffraction, includes a spinel phase and optionally a zinc oxide phase, the sum of which accounts for in the interval Q-100% by weight of all oxidic phases in the catalyst, where Q depends on the maximum calcination temperature the catalyst has been exposed to in air for a period of in the interval 1-10 hours (Tcalc), so that
g1) if 250° C. Tcalc 550° C., then Q=80, preferably Q=90, more preferably Q=95, most preferably Q=99
g2) if 550° C.≤Tcalc≤900° C., then Q=50, such as Q=60
h) having a percentage Z of visible CuO, defined as the percentage Wt % CuO according to XRD relative to the maximum possible Wt % CuO calculated from bulk elemental analysis (ICP or similar method), where Z depends on the maximum calcination temperature the catalyst has been exposed to in air for a period of in the interval 1-10 hours (Tcalc), so that 0<Z<0.125*Tcalc, where the unit of Tcalc is ° C.

Embodiment 2. A process according to embodiment 1, wherein said catalyst has been exposed to a temperature Tcalc of between 300-900° C., preferably between 450-750° C.

Embodiment 3. A process according to any one of embodiments 1 or 2, wherein said catalyst has been exposed to a calcination temperature Tcalc in the range of 550-700° C.

Embodiment 4. The catalyst according to any one of embodiment 1 to 3, wherein tablets of said catalyst in its oxidized form have a radial crush strength, SCS, of between 25 and 150 kp/cm, said tablets having a tablet density in the range of 1.45-2.35 g/cm³, preferably in the range of 1.65-2.35 g/cm³.

Embodiment 5. The catalyst according to any one of embodiments 1 to 3, wherein tablets of said catalyst in its freshly reduced form have a radial crush strength of between 10 and 75 kp/cm, said tablets having a tablet density in the interval 1.45-2.35 g/cm³, preferably in the range 1.65-2.35 g/cm³.

1. A catalyst composition for catalytic hydrogenation of an organic carbonyl compound, the composition comprising in its oxidized form 12-38% by weight of Cu, 13-35% by weight of Zn, and 12-30% by weight of Al;
and the composition having a molar ratio of Zn:Al in the range of from 0.24-0.60;
and the composition comprising in its oxidized form at least 50% by weight of a spinel structure as determined by X-ray diffraction (XRD).

2. The catalyst composition according to claim 1 having a molar ratio of Zn:Al in the range of from 0.30-0.55.

3. The catalyst composition according to claim 1, wherein the composition comprises in its oxidized form at least 60% by weight of a spinel structure as determined by X-ray diffraction.

4. The catalyst composition according to claim 1, wherein the catalyst composition comprises in its oxidized form 15-38% by weight of Cu.

5. The catalytic composition according to claim 1 having in its oxidized form an olive-green color corresponding to approximately Red:100 Green:100 Blue:50.

6. The catalyst composition according to claim 1, wherein the catalyst composition comprises in its oxidized form 13-24% by weight of Zn.

7. The catalyst composition according to claim 1, wherein the catalyst composition comprises in its oxidized form 17-24% by weight of Al.

8. The catalyst composition according to claim 1, wherein the catalyst composition in its oxidized form comprises less than 0.01 Wt % Ni and/or less than 0.01 Wt % Cr.

9. The catalyst composition according to claim 1, having in its oxidized form a radial crush strength, SCS, of between 25 and 150 kp/cm and/or a density in the range of from 1.45-2.35 g/cm3.

10. The catalyst composition according to claim 1, having in its reduced form a radial crush strength, SCS, of between 10 and 75 kp/cm, and/or a density in the range of from 1.45-2.35 g/cm3.

11. The catalyst composition according to claim 1 having in its reduced form a copper metal surface area above 10 m²/g Cu.

12. The catalyst composition according to claim 1 comprising in its oxidized form less than 15% by weight of ZnO.

13. A method for preparing an oxidized form of a catalyst composition for catalytic hydrogenation of an organic carbonyl compound comprising the steps of:
a. Coprecipitating:
I. an acidic solution of salts of Cu and Zn having a Cu:Zn weight ratio in the range of from 0.3-2.5; and
II. a basic solution of an aluminate salt further containing one or more soluble hydroxide salts and one or more soluble carbonate salts;
to obtain a catalyst precursor composition having a molar ratio of Zn:Al in the range of from 0.24-0.60;
b. Calcining the catalyst precursor composition at a temperature Tcalc in the range of from 250-900° C. to obtain an oxidized form of a catalyst composition for catalytic hydrogenation of an organic carbonyl compound, the catalyst composition comprising in its oxidized form 12-38% by weight of Cu, 13-35% by weight of Zn, and 12-30% by weight of Al, the remainder being mainly oxygen; and the catalyst composition having a molar ratio of Zn:Al in the range of from 0.24-0.60; and the catalyst composition comprising in its oxidized form at least 50% by weight of a spinel structure as determined by X-ray diffraction (XRD).

14. The method according to claim 13, wherein the calcination of step b) is conducted for a period of time in the range of from 1-10 hours.

15. The method according to claim 13, wherein the catalyst precursor composition of step a) is tableted prior to the calcination of step b).

16. The method according to claim 13, wherein the calcination of step b) of the catalyst precursor composition is conducted at a temperature Tcalc in the range of from 300-900° C.

17. The method according to claim 13, wherein the aluminate salt of step a.ii. is provided as an alkali aluminate selected from the group consisting of lithium aluminate, sodium aluminate, potassium aluminate, rubidium aluminate and cesium aluminate.

18. The method according to claim 13, wherein the pH of the coprecipitation step a. is in the range of 6-12.

19. A catalyst composition in its oxidized form obtainable by claim 13, and suitable for catalytic hydrogenation of an organic carbonyl compound.

20. A catalyst precursor composition obtainable by step a. of claim 13 suitable for preparing a catalyst composition in its oxidized form for catalytic hydrogenation of an organic carbonyl compound.

21. A catalyst composition in its reduced form obtainable by reducing the catalyst composition according to claim 1, and suitable for catalytic hydrogenation of an organic carbonyl compound.

22. A process for hydrogenating a carbonyl group of an organic carbonyl compound into its corresponding hydroxyl group, the process comprising contacting the organic carbonyl compound with a reduced form of the catalyst composition according to claim 1 in the presence of hydrogen to obtain an alcohol corresponding to said organic carbonyl compound.

23. The process according to claim 22, wherein the hydrogenation is conducted at a temperature of from 150-300° C.

24. The process according to claim 22, wherein the carbonyl compound is selected from the group comprising formaldehyde, glycolaldehyde, glyoxal, pyruvic aldehyde, acetol, and butyraldehyde.

25. A use of a catalyst according to claim 1 for hydrogenation of a feed comprising at least two of the carbonyl compound selected from the group comprising formaldehyde, glycolaldehyde, glyoxal, pyruvic aldehyde and acetol.

26. The use according to claim 25, wherein the hydrogenation is a gas phase hydrogenation.

27. A use of alkali aluminate, such as potassium aluminate or sodium aluminate, for preparing a catalyst composition in its oxidized form or its reduced form for hydrogenation reactions.
